(19) United States
(12) Patent Application Publication
(10) Pub. No.: US 2012/0077786 A1
(43) Pub. Date: Mar. 29, 2012

(54) METHODS AND COMPOSITIONS FOR DISEASE TREATMENT USING INHALATION

(76) Inventors: David A. Byron, Lawrenceville, NJ (US); Alan B. Watts, Austin, TX (US); Robert O. Cook, Hillsborough, NJ (US); Murat Aydin, North Brunswick, NJ (US)

(21) Appl. No.: 13/246,686

(22) Filed: Sep. 27, 2011

Related U.S. Application Data
(60) Provisional application No. 61/386,733, filed on Sep. 27, 2010.
(60) Provisional application No. 61/386,767, filed on Sep. 27, 2010.
(60) Provisional application No. 61/386,771, filed on Sep. 27, 2010.
(60) Provisional application No. 61/386,776, filed on Sep. 27, 2010.

(51) Int. Cl. A61K 31/593; A61P 11/00

(52) U.S. Cl. 514/167

(57) ABSTRACT

Methods and compositions for the treatment of pulmonary disease using inhalation are provided. In particular, the present disclosure provides novel methods and compositions for treating pulmonary diseases such as asthma, bronchitis, COPD, emphysema, lung cancer, pneumonia and pulmonary edema. In addition, the present disclosure provides novel methods and compositions for treating complications associated with pulmonary disease such as corticosteroid resistance and pulmonary tissue destruction. The compositions of the present disclosure comprise corticosteroid resistance agents including but not limited to vitamin D, calcitriol and equivalents thereof. The compositions of the present disclosure also comprise alveolar development and maintenance agents including but not limited to vitamin A, ATRA and equivalents thereof. The present invention provides effective administration of therapeutic agents to specific airways of the lungs by utilizing controlled site delivery.

### CROSS REFERENCE TO RELATED APPLICATION

This application claims priority to U.S. Provisional Patent Application Ser. No. 61/386,733 filed on Sep. 27, 2010, U.S. Provisional Patent Application Ser. No. 61/386,767 filed on Sep. 27, 2010, U.S. Provisional. Patent Application Ser. No. 61/386,771 filed on Sep. 27, 2010, U.S. Provisional Patent Application Ser. No. 61/386,776 filed on Sep. 27, 2010, the contents of which are incorporated herein in their entirety, by reference.

### FIELD OF THE INVENTION

The present invention relates generally to the field of treating pulmonary diseases comprising the administration of therapeutic agents using inhalation devices. The disclosure has particular utility in connection with the delivery of powdered medications to a patient, and will be described in connection with such utility, although other utilities are contemplated. More specifically, the present invention relates to novel dosage forms and compositions for treating pulmonary diseases, including but not limited to, complications such as corticosteroid resistance. In certain embodiments, the present invention is also related to improving underlying physiological dysfunction contributing to pulmonary disease. The present invention provides effective administration of therapeutic agents to specific airways of the lungs by utilizing controlled site delivery.

### BACKGROUND OF THE INVENTION

There exists a significant need for efficient inhalation devices that deliver medicaments for individuals suffering from pulmonary disease. Patients inflicted with pulmonary problems such as asthma, emphysema or chronic obstructive pulmonary disorder, are often faced with challenges in administering therapeutic agents sometimes resulting in life-threatening complications. An individual suffering from difficulties associated with breathing may be further stressed by having to receive his or her medication via inhalation due to blocked airway passages. Optimal delivery via inhalation nevertheless remains the preferred mechanism of treatment for such patients as controlled site delivery, i.e. delivery of therapeutic agents to the lungs, airway passages, bronchioles, and alveoli, is the most efficient way in which to deliver medication and alleviate symptoms.

Pulmonary disease, or lung disease, is any disease or disorder that causes the lungs not to function properly. There are three main types of pulmonary/lung diseases and they are generally categorized as airway diseases, lung tissue diseases, and lung circulation diseases.

Airway diseases affect the tubes (airways) that carry oxygen and other gases into and out of the lungs. These diseases usually cause a narrowing or blockage of the airways. They include asthma, emphysema, and chronic bronchitis. People with airway diseases sometimes describe the feeling as “trying to breathe out through a straw.” Lung tissue diseases affect the structure of the lung tissue. Scarring or inflammation of the tissue makes the lungs unable to expand fully (“restrictive lung disease”). This makes it hard for the lungs to breathe in oxygen and release carbon dioxide. Pulmonary fibrosis and sarcoidosis are examples of lung tissue diseases. People sometimes describe the feeling as “wearing a sweater or vest that is too-tight” that won't allow them to take a deep breath. Lung circulation diseases affect the blood vessels in the lungs and they are caused by clotting, scarring, or inflammation of the blood vessels. Lung circulation diseases consequently affect the ability of the lungs to take up oxygen and to release carbon dioxide and may also affect heart function.

Pulmonary disease includes, but is not limited to, acute bronchitis, acute respiratory distress syndrome (ARDS), asbestosis, asthma, atelectasis, aspergilliosis, bronchiectasis, bronchiolitis, bronchopulmonary dysplasia, byssinosis, chronic bronchitis, coccidiomycosis, chronic obstructive pulmonary disease (COPD), cystic fibrosis, emphysema, eosinophilic pneumonia, hantavirus pulmonary syndrome, histoplasmosis, human metapneumovirus, hypersensitivity pneumonitis, influenza, lung cancer, lymphangiomatosis, mesothelioma, necrotizing pneumonia, nontuberculosis Mycobacterium, pertussis, pleural effusion, pneumoconiosis, pneumonia, primary ciliary dyskinesia, primary pulmonary hypertension, pulmonary arterial hypertension, pulmonary fibrosis, pulmonary vascular disease, respiratory syncytial virus, sarcoidosis, severe acute respiratory syndrome, silicosis, sleep apnea, sudden infant death syndrome, and tuberculosis. The most common lung diseases generally comprise asthma, bronchitis, COPD, emphysema, lung cancer, pneumonia and pulmonary edema.

Of all pulmonary diseases, the most prevalent appears to be COPD. According to the World Health Organization estimates in the year 2004, 64 million people had COPD and 3 million people died of COPD. WHO predicts that COPD will become the third leading cause of death worldwide by 2030. The Merck Manual (2011) provides that an estimated 12 million people in the US have COPD and describes COPD as the 4th leading cause of death, resulting in 122,000 deaths in 2003 compared with 52,193 deaths in 1980. From 1980 to 2000, the COPD mortality rate increased 64% (from 40.7 to 66.9/100,000). Prevalence, incidence, and mortality rates increase with age and though prevalence is higher in men, total mortality is similar in both sexes. Incidence and mortality are generally higher in caucasians, blue-collar workers, and people with fewer years of formal education, probably because these groups have a higher prevalence of smoking. COPD is increasing worldwide because of the increase in smoking in developing countries, the reduction in mortality due to infectious diseases, and the widespread use of biomass fuels.

COPD is partially reversible airflow limitation caused by an inflammatory response to inhaled toxins, often cigarette smoke, α₁-Antitrypsin deficiency and various occupational exposures. Symptoms are productive cough and dyspnea that develop over years; common signs include decreased breath sounds, prolonged expiratory phase of respiration, and wheezing. Severe cases may be complicated by weight loss, pneumothorax, frequent acute decompensation episodes, right heart failure, and acute or chronic respiratory failure. Diagnosis is based on history, physical examination, chest x-ray, and pulmonary function tests. Treatment is with bronchodilators, corticosteroids, and, when necessary, O₂ and antibiotics. About 50% of patients die within 10 years of diagnosis.

COPD is also manifested outside of the airways by extra-pulmonary inflammation and muscular atrophy. COPD is a heterogeneous disease encompassing inflammation and excessive mucus secretion in the large and small airway as well as destruction of the alveolar sacs. Airway remodeling occurs as a result of inflammation associated with emphysema, leading to disruption in the alveolar attachment of the small airways and subsequent airway closure during exhalation (as alveolar attachments are no longer able to hold the airway open). Disease progression leads to air trapping, hyperinflation and reduced inspiratory capacity.

COPD comprises chronic obstructive bronchitis (clinically defined) and emphysema (pathologically or radiologically defined), and many patients have features of both.

Chronic obstructive bronchitis is chronic bronchitis with airflow obstruction. Chronic bronchitis is defined as productive cough on most days of the week for at least three months total duration in two successive years. Chronic bronchitis becomes chronic obstructive bronchitis if spirometric evidence of airflow obstruction develops. Chronic asthmatic bronchitis is a similar, overlapping condition characterized by chronic productive cough, wheezing, and partially reversible airflow obstruction; it occurs predominantly in smokers with a history of asthma. In some cases, the distinction between chronic obstructive bronchitis and chronic asthmatic bronchitis is unclear.

Emphysema is destruction of lung parenchyma leading to loss of elastic recoil and loss of alveolar septa and radial airway traction, which increases the tendency for airway collapse. Lung hyperinflation, airflow limitation, and air trapping follow. Airspaces enlarge and may eventually develop bullae.

Current therapeutic agents for COPD predominately comprise bronchodilators administered via inhalation, including inhaled long-acting beta₂-agonists (LABA) or long acting muscarinic antagonists (LAMA). Although corticosteroids have been proven effective in other inflammatory diseases such as asthma, rheumatoid arthritis, and ulcerative colitis, their use is often ineffective in COPD outside of exacerbation reduction, leading some to question their importance as a therapeutic in the disease. Conversely, combinations of bronchodilators with long acting corticorsteroids have found utility in preventing COPD exacerbations and treating contaminant asthma, but the utility of corticosteroids alone have not been demonstrated. Despite being unable to fully address the inflammation and destructive process associated with the progression of COPD, various double and triple combination products using corticosteroids are in development.

### Corticosteroid Resistance

There has been much investigation into the mechanisms of corticosteroid resistance, many of which are based on an underexpression of chemical mediators involved in the regulation of inflammation in COPD. It has also been suggested that the seemingly “resistant” nature of COPD toward inhaled corticosteroid therapy (ICS) may not only be due to a physiological resistance, but may reflect the lack of drug deposition in the small airways. In a study investigating the efficacy of extrafine beclomethasone dipropionate (1.1 microns in diameter using a HFA pMDI) in patients with COPD, a significant reduction in air trapping was measured, suggesting a reduction in small airway inflammation. No prior art dry powder inhalation devices have the ability to deliver extrafine particles in patients with COPD. What is needed is a method of drug delivery that effectively targets the small airways and lung parenchyma, which are the sites of inflammation for pulmonary disease such as COPD. Current therapies lack the capability to achieve high levels of small airway deposition due to a number of issues:
(1) The mass median aerodynamic diameter (MMAD) is too large and geometric standard deviation (GSD) is too broad to effectively target the small airway.
(2) Delivery devices require minimal flow rates for optimal operation that are often beyond the capability of severely flow-restricted subjects.
(3) Delivery devices require patient coordination which proves difficult for elderly patients, or patients having compromised physical abilities.
(4) Aerosols are partially blocked or blocked by collapsed airways.
(5) Aerosols produced near the end of the inspiratory breath will not have sufficient time to deposit in the small airways before exhalation.

Regardless of whether ICS is effective or ineffective, it is clear that the nature of the inflammation in COPD is less responsive to the current ICS therapies than other inflammatory diseases such as asthma, rheumatoid arthritis, and ulcerative colitis, and requires a different approach to immunomodulatory therapy. Corticosteroid resistance may be due in part to the limitation of effective therapeutic delivery mechanisms involving the ability (or lack thereof) to deposit medicaments within the small airway passages of the lungs, however evidence is also emerging supporting the contention that metabolic and physiological malfunction may manifest in conditions that prevent COPD patients from responding to corticosteroid therapy.

One mechanism thought to lead to corticoidsteroid resistance (CR) in patients with COPD pertains to the reduced expression of histone deacetylase (HDAC) within an inflammatory cell. HDAC is normally recruited by activated glucocorticoid receptors and results in deacetylation and “switching off” of genes transcribing for inflammatory cytokines and chemokines. Most COPD patients, being unresponsive to corticosteroids, possess lower levels of HDAC, and thus are more prone to severe inflammation. It is suspected that oxidative and nitrative stresses, which are commonly found in cigarette smoke, are the primary reasons for inhibition of HDAC in COPD. In a study investigating the influence of HDAC on corticosteroid-resistant bronchoalveolar macrophages, it was found that corticosteroid sensitivity could be increased when HDAC was overexpressed.

CR is also thought to occur when there is a lack of IL-10 secretion from regulatory T cells. IL-10 plays an important role in the downregulation of Th1 inflammatory cytokines and promotion of regulatory T cells which help to control the inflammatory response. In a study investigating the response of CR CD4+ T cells to calcitriol and dexamethasone, it was found that co-administration of these agents to cell lines increased IL-10 levels to those seen in normal, corticosteroid-sensitive cell lines. Accordingly, recent studies support the theory that CR may be the result of physiological changes manifested at the molecular level and likely induced by pulmonary trauma such as that caused by cigarette smoking or other oxidative stress. Nevertheless, though the role of CR resistance has been identified, no effective therapeutic means or strategies are available for reducing or reversing COPD-related consequences of CR resistance.

### Vitamin D and Pulmonary Disease

Vitamin D is a lipophilic small molecule responsible for maintaining normal calcium metabolism in the body. It encompasses several vitamers including vitamin D₁, vitamin D₂, vitamin D₃, vitamin D₄, and vitamin D₅. Cholecalciferol (vitamin D₃) is the animal-derived form of vitamin D and is produced in the skin when ultraviolet radiation cleaves the steroidal ring of 7-dehydrocholestorol. In humans, the majority of cholecalciferol is maintained by sun light exposure; however, it may also be supplemented to some extent by dietary consumption. Hepatic metabolism of cholecalciferol gives rise to the most prevalent circulating metabolite, calcidiol (25-hydroxyvitamin D₃), which is in turn metabolized by the kidney to form the most physiologically active vitamin D metabolite, calcitriol (1,25-dihyroxyvitamin D₃). Synthetic versions of calcitriol have been produced by the pharmaceutical industry, as well as other synthetic activators of the vitamin D receptor doxercalciferol and paricalcitol.

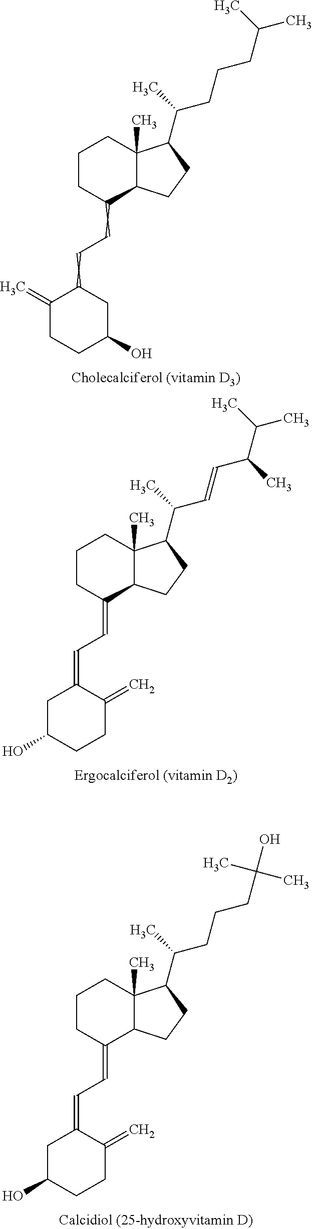

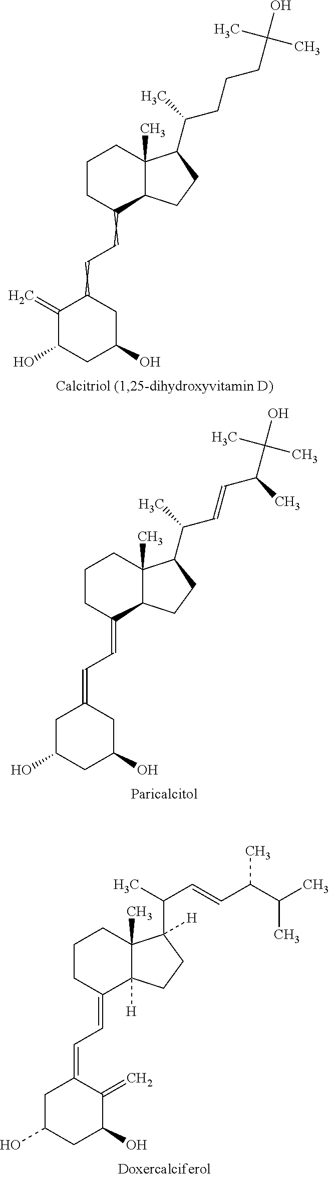

The immunomodulatory effects of vitamin D have been well described in the literature and are predominantly due to its most active metabolite, calcitriol. Calcitirol acts on a variety of inflammatory cells including monocytes, macrophages, dendritic cells, effector T cells, and B cells and in turn affects the expression genes encoding for chemical mediators of inflammation. T_{H}1 associated cytokines, such as IL-2, IL-6, IL-8, IL-12, and IFNγ, are generally downregulated by calcitriol and may lead to a more T_{H}2 mediated inflammation through the upregulation of IL-4 (Mora et al. Nat. Rev. Immunol. 8(9) (2008) 685-698). However, there is evidence showing that T_{H}2-associated pulmonary inflammation may also be reduced (as measured through a reduction in IL-5 and eosinophils in bronchioalveolar lavage fluid), despite the apparent shift toward T_{H}2 cytokine expression systemically (Sandhu et al. American College of Allergy, Asthma, & Immunology 105(3) 191-199). While the anti-inflammatory mechanisms behind these seemingly contradictory findings remain unclear, the upregulation of IL-10, an anti-inflammatory cytokine inhibiting T_{H}1 and T_{H}2 responses, could be attributed to calcitirol's broad inflammation reduction effects. Calcitirol has been shown to promote IL-10 secreting regulatory T cells as well as IL-10 expression by dendritic cells in preclinical models. An in vitro study of a cell line cultured from patients with corticosteroid resistant asthma, promotion of regulatory T cells after addition of vitamin D resulted in increased steroid sensitivity, suggesting that vitamin D may be able to reverse corticosteroid resistance (Xystrakis et al. The Journal of Clinical Investigation 116(1) (2006) 146-155). Despite studies by Xystrakis et al. and others, effective vitamin D therapy resulting in the reversal of corticosteroid resistance has not been accomplished or reduced to practice.

Vitamin D has been demonstrated to play an important role in improved lung function. A trial in asthmatic adults with varying levels of vitamin D has shown that a 22.7 mL mean increase in FEV₁ can be expected for every 1 ng/mL increase in systemic vitamin (D Sutherland et al. Am. J. Respir. Crit. Care Med. 181(7) (2010) 699-704). In a study enrolling 100 asthmatic children, steroid dose used for asthma maintenance therapy was inversely proportional to systemic vitamin D levels. It was also found that vitamin D levels were directly proportional to FEV1 and inversely proportional of circulating IgE concentrations (The Journal of Allergy and Clinical Immunology 125(5) (2010) 995-1000).

Chronic asthma and COPD often result in airway remodeling which is detrimental to lung function and limits a patient's quality of life. This aspect of airway disease has also been shown responsive to vitamin D therapy. Airway smooth muscle proliferation, a contributing factor in airway remodeling seen in severe asthma and COPD, has been shown to slow due to the anti-proliferative effects of vitamin D Damera et al. Am. J. Respir. Crit. Care Med. 179 (2009) A5606. Currently, several clinical trials are underway to investigate the effect of oral supplementation of vitamin D on asthma and COPD, however no such trials or studies have been successfully accomplished for inhaled supplementation of vitamin D.

In addition to its immunomodulating and pulmonary effects, there is evidence that vitamin D may also function as an anti-proliferative, effective against cancer. The anti-tumor effects of vitamin D are multifaceted and most likely due to the arrest of Gₒ/G₁ phase of the cell cycle, induction of apoptosis, inhibition of cell growth, and induction of cell differentiation in malignant cells. The activity of vitamin D toward a variety of cancer lines (prostate, breast, colorectal, head/neck, lung) is attributed to the presence of the vitamin D receptor (VDR) in the cell membranes of these malignant cell types. In situations of vitamin D deficiency, generally due to reduction in sunlight exposure and genetic factors, incidence rate of some cancers have been shown to increase, further implicating the role of vitamin D in normal physiological anti-cancer functions.

In clinical trials investigating the use of calcitriol as an anti-cancer therapy, very high doses of calcitriol are required to impart a therapeutic effect, leading to concern over toxicity due to hypercalcemia Urologic Oncology 21(5) (2003) 399-405. There is evidence that dose limitation may not be solely attributed to toxicity, but may be a result of limited absorption or orally dosed calcitriol. Alternative methods of delivery are necessary to achieve greater bioavailability through the avoidance of intestinal absorption and first pass metabolism, thus limiting or eliminating potential toxicity problems resulting from high doses of calcitriol.

Not only does vitamin D have a potential therapeutic role in pulmonary diseases including asthma, COPD, pulmonary infection and lung cancer, vitamin D therapy is important for osteoporosis, hypocalcemia, hyperparathyroidism and cancer. Accordingly, there exists a need for effective dosing and administration of vitamin D wherein toxic side effects are reduced and preferably eliminated. Preferably, such dosing and administrative means should be easy to handle and safe for the user to receive the prescribed amount of vitamin D in a form that is metabolically and physiologically appropriate.

### Vitamin A and Alveolar Development

Vitamin A is important for lung development and lung function through generating alveolar septa which are capable of gas exchange. These effects are mediated by the Retinoic Acid Receptor [RAR] gamma subtype in alveolar walls, and are triggered by All Trans Retinoic Acid (ATRA), which is the active metabolite of Vitamin A. Exogenous ATRA can influence the formation of alveoli in newborn and adult rodents Am J Physiol: Lung Cell and Mol. Physiol. 2004 286; 2: ppL249-256. Furthermore, ATRA treatment of adult rats with preexisting elastase-induced emphysema induces alveolus formation returning the size, number, and surface area of alveoli, and tissue elastic recoil, to values present in same-aged rats not treated with elastase (Nat Med 1997 3: pp 675-677). These effects are governed by the effects of the RAR on gene expression. ATRA diminishes the formation of pulmonary emphysema in mice exposed to cigarette smoke and decreases the distance between alveolar walls in mice with emphysema produced by cigarette smoke. It therefore follows that ATRA therapy, or therapy with RAR specific agents has the possibility to treat COPD, COPDe and emphysema by generating new alveoli for greater gas exchange, however no such therapies are currently available.

None of the above models, however accurately represent human emphysema. In fact, higher order species have shown less clear results with the use of ATRA on alvelogenesis (Am J Respir Cell Mol Biol 2002 26: pp 52-57) and recent trials in adults with hereditary emphysema with ATRA (Chest 2006 130; 5: pp 1334-1345) and RAR-gamma selective agents have not been successful. Elsewhere in the body, ATRA is known to induce matrix-metalloproteinase-9 (MMP-9) and Interleukin-8 which are likely additive to the inflammatory cascade in COPD and emphysema and cause progressive loss of pulmonary function and likely destruction of newly formed alveoli (Br J. Haematol. 2002 118; 2: pp 419-25). Accordingly, though studies have investigated the effects of ATRA, the active metabolite of vitamin A, on pulmonary function and alveolar formation, the findings are inconsistent and currently no therapeutic formulations or mechanisms are available that effectively deliver vitamin A with the ultimate goal of improving lung function and decreasing COPD or other pulmonary malfunction.

Treatment of Respiratory Disease using Inhaled Therapeutics

Many diseases of the respiratory tract are known to respond to treatment by the direct application of therapeutic agents. As these agents are most readily available in dry powdered form, their application is most conveniently accomplished by inhaling the powdered material through the nose or mouth. This powdered form can result in the better utilization of the medicament in that the drug is deposited exactly at the site desired and where its action may be required; hence, very minute doses of the drug are often equally as efficacious as larger doses administered by other means, with a consequent marked reduction in the incidence of undesired side effects and medicament cost. In addition, a drug in dry powder form may be used for treatment of diseases other than those of the respiratory or pulmonary system. When the drug is deposited on the very large surface areas of the lungs, it may be very rapidly absorbed into the blood stream; hence, this method of application may take the place of administration by injection, tablet, or other conventional means.

It is the opinion of the pharmaceutical industry that the bioavailability of the drug is optimum when the drug particles delivered to the respiratory tract are between 1 to 5 microns in size. When drug particles need to be in this size range, dry powder delivery systems need to address a number of issues:
(1) Small size particles may develop an electrostatic charge on themselves during manufacturing and storage. This may cause the particles to agglomerate or aggregate, resulting in clusters of particles which have an effective size greater than 5 microns. The probability of these large clusters navigating to the deep lungs then decreases. This in turn results in a lower percentage of the packaged drug being available to the patient for absorption.
(2) The amount of active drug that needs to be delivered to the patient may be of the order of 10s of micrograms. For example, in the case of albuterol, a drug used in asthma, this is usually 25 to 100 micrograms. Current manufacturing equipment cannot effectively deliver aliquots of drugs in milligram dose range with acceptable accuracy. So the standard practice is to mix the active drug with a filler or bulking agent such as lactose. This additive also makes the drug “easy to flow”. This filler is also called a carrier since the drug particles also stick to these particles through electrostatic or chemical bonds. These carrier particles are very much larger than the drug particles in size. The ability of an inhaler to separate drug from the carrier is an important performance parameter in the effectiveness of the design.
(3) Active drug particles with sizes greater than 5 microns will be deposited either in the mouth or throat. This introduces another level of uncertainty since the bioavailability and absorption of the drug in these locations is different from the lungs. Inhalers need to minimize the drug deposited in these locations to reduce the uncertainty associated with the bioavailability of the drug.

Three types of inhaler devices have been traditionally used to create the aerosol needed for pulmonary delivery: dry powder inhalers (DPIs), metered dose inhalers (MDIs), and aqueous nebulizers.

### Dry Powder Inhalers

Prior art dry powder inhalers (DPIs) usually have a means for introducing the drug (active drug plus carrier) into a high velocity air stream. The high velocity air stream is used as the primary mechanism for breaking up the cluster of micronized particles or separating the drug particles from the carrier. Several inhalation devices useful for dispensing powder forms of medicament are known in the prior art. For example, in U.S. Pat. Nos. 3,507,277; 3,518,992; 3,635,219; 3,795,244; and 3,807,400, inhalation devices are disclosed having means for piercing of a capsule containing a powdered medicament, which upon inhalation is drawn out of the pierced capsule and into the user's mouth. Several of these patents disclose propeller means, which upon inhalation aid in dispensing the powder out of the capsule, so that it is not necessary to rely solely on the inhaled air to suction powder from the capsule. For example, in U.S. Pat. No. 2,517,482, a device is disclosed having a powder containing capsule placed in a lower chamber before inhalation, where it is pierced by manual depression of a piercing pin by the user. After piercing, inhalation is begun and the capsule is drawn into an upper chamber of the device where it moves about in all directions to cause a dispensing of powder through the pierced holes and into the inhaled air stream. U.S. Pat. No. 3,831,606 discloses an inhalation device having multiple piercing pins, propeller means, and a self-contained power source for operating the propeller means via external manual manipulation, so that upon inhalation the propeller means aids in dispensing the powder into the stream of inhaled air. See also U.S. Pat. Nos. 3,948,264 and 5,458,135.

In prior U.S. Pat. Nos. 7,318,434 and 7,334,577 incorporated herein by reference, and assigned to the common assignee MicroDose Technologies, Inc., there is provided an improvement over prior art inhalers that utilize vibration to facilitate suspension of power into an inhaled gas stream and which utilizes synthetic jetting to aerosolize drug powder from a blister pack or the like. As taught in the aforesaid U.S. Pat. Nos. 7,318,434 and 7,334,577 there is provided a dry powder inhaler having a first chamber such as a blister pack or other container, for and holding a dry powder, and a second chamber connected to the first chamber via a passageway for receiving an aerosolized form of the dry powder from the first chamber and for delivering the aerosolized dry powder to a user. A vibrator is coupled to the dry powder in the first chamber. The vibrator is energized and coupled to the first chamber and drives the powder from the chamber by synthetic jetting.

As described in U.S. Pat. No. 7,080,644 also incorporated herein by reference, and also assigned to common assignee MicroDose Technologies, Inc., controlled aliquots or doses of a medication or drug are pre-packaged in a blister pack, which includes a frangible crowned top element which may be conical, conical with a rounded point, rounded, or other raised shape configuration, and a bottom element which may be a flat web or membrane, or which itself may be of shaped configuration, e.g, conical, round, dish shaped, etc. for closely engaging with an underlying vibrating element, the shape and size of which is chosen to provide optimum controlled delivery of a given medication or drug. The top element of the blister pack is pierced with a piercing device such as a sharp needle to form one or more apertures for delivery of the medication or drug contained within the blister pack. The hole pattern and hole size is selected to provide optimization of delivery of the particular medication or drug packaged therein.

### Metered Dose Inhalers

Metered dose inhalers (MDIs) have a pressurized canister filled with a liquid propellant. The drug is either suspended or dissolved in the propellant. The MDIs have a metering valve for metering out a known quantity of the propellant and hence the drug. When the canister is depressed against the MDI housing a known quantity of the propellant is discharged. The propellant evaporates leaving behind a fine aerosol of the drug suitable for inhalation by the patient. For effective delivery of the drug to the lungs the patient needs to coordinate breath inhalation with the discharge of the drug from the canister. Patients are not always effective in achieving this coordination leading to dose variability. Incorporation of a breath actuation mechanism addresses this concern but the variability still exists because of the “cold” freon effect where the patient stops breathing when the cold aerosol hits the back of the throat. This is especially true of the pediatric patients where co-ordination is of major concern. To overcome these limitations and to minimize the variability of the dose delivered, the MDI is normally recommended to be used with a spacer especially for children. The primary function of the spacer is to slow down the MDI discharge and function as a holding chamber for the aerosol plume. A face mask may be attached to the end of the spacer. These spacers normally are made of plastic and therefore tend to build up electrostatic charge on the inside surface of the spacer. The large dead space between the inlet and outlet of the spacer coupled with the electrostatic charge has the effect of lowering the amount of dose delivered and the amount of drug that is in the respirable range. It is estimated that MDIs deliver about 10% to 20% of the dose to lungs in adults with good coordination. Studies have shown that for pediatric patients between the ages of 3 years to 5 years using an MDI with a spacer and face mask, the lung delivery is less than 10% of the dose. Accordingly, drug delivery using current MDIs is ineffective, especially among pediatric patients.

### Nebulizers

Nebulizers, such as the jet nebulizers, produce a fine aerosol mist/droplets which carry the drug either as a suspension or dissolved in the aqueous medium. The jet nebulizers use compressed air to atomize the aqueous solution. The flow rate of the compressed air should be matched to the inhalation flow rate of the patient for optimum delivery of the drug. A drug can be administered to a patient with repetitive non-forced inhalation over a prolonged period of time. The amount of drug delivered is influenced by a large number of factors such as viscosity, volume of drug fill, surface tension, inhalation flow, etc. The amount of drug delivered ranges from 3% to 6% for pediatric patients and 3% to 13% for adults. Pediatric delivery nebulizers are normally coupled to a face mask. Since the nebulizer continues to produce the aerosol during the exhale cycle of the breath this leads to drug wastage, increased exposure of the drug to the patient's face and eyes and also to the caregiver. The disadvantages of nebulizers in general are their poor efficiency of delivery to the patient, a requirement for a compressor or compressed air and long delivery times, on the order of 5 to 20 minutes.

Thus there is a need for a delivery mechanism for infants and young children, and also for respiratory compromised patients that overcomes the aforesaid and other disadvantages of the prior art, in a manner that delivers the drug efficiently, does not require inhalation coordination, operates under low inhalation volume, minimizes the exposure of the caregiver to the drug, delivers the drug in a short time (preferably less than a minute), and is low cost and portable.

What is needed therefore, is an improved and efficient method and delivery device for depositing therapeutic agents within the pulmonary cavities of affected subjects, wherein in such therapeutic agents include those suited to address complications associated with corticosteroid resistance. More specifically, methods and delivery devices that successfully result in the deposition of therapeutic agents within the small airways and lung parenchyma are particularly desirable. Such methods and devices should be easy to administer and facilitate therapeutic compliance. In addition, such methods and devices should preferably be available for all relevant indications and not be limited to those related to pulmonary disease and malfunction.

### SUMMARY OF THE INVENTION

The present disclosure provides an improvement over prior art devices such as discussed above by providing methods for treating pulmonary disease comprising the use of improved inhaler devices for the delivery of therapeutic compositions via inhalation. The improved methods of the present invention satisfy the heretofore unmet need in the art for methods and devices that enable the efficient deposition of therapeutic and pharmaceutical agents to the small airways and parenchyma of the lungs. The inhalers combine the properties of controlling the drug particle size as well as the dosing mechanism by which the drug is delivered to the subject.

In addition, the methods of the present invention are particularly useful for addressing complications associated with pulmonary disease including, but not limited to, corticosteroid resistance (CR), as the devices used herein have the functionality to deliver drugs (such as CR reversal agents) deep into the lung tissues, and may also be configured to deliver more than one therapeutic agent (i.e. CR reversal agent and corticosteroid). The methods and compositions of the present invention may be further utilized for addressing physiological and anatomical destruction associated with pulmonary malfunction; for example, in certain embodiments, the methods and compositions of the present invention may be targeted to improving alveolar function and development via the administration of alveolar regrowth and/or maintenance agents

The methods and compositions described herein are particularly suited for depositing therapeutic agents necessary for alleviating symptoms associated with pulmonary disease and malfunction, however, as would be evident to one skilled in the art, they may also be utilized for additional indications.

Accordingly, it is an object of the present invention to provide improved methods and devices for the delivery of therapeutic and pharmaceutical agents to the small airways and parenchyma of the lungs.

Another object of the present invention is to provide improved methods and devices for the delivery of therapeutic and pharmaceutical agents to the small airways and parenchyma of the lungs, wherein such devices combine controlling drug particle size and delivery mechanism to optimize delivery.

Another object of the present invention is to provide improved methods and devices for the delivery of therapeutic and pharmaceutical agents to the small airways and parenchyma of the lungs, wherein such devices are self-contained, easy to use, and improve therapeutic compliance.

Yet another object of the present invention is to provide improved methods and devices for the delivery of therapeutic and pharmaceutical agents to the small airways and parenchyma of the lungs, wherein such devices overcome the limitations of patients having restricted inspiratory flow.

Yet another object of the present invention is to provide improved methods and devices for the delivery of therapeutic and pharmaceutical agents to the small airways and parenchyma of the lungs, wherein the therapeutic and pharmaceutical agents alleviate symptoms associated with pulmonary disease and malfunction.

A further object of the present invention is to provide improved methods and devices for the delivery of therapeutic and pharmaceutical agents to the small airways and parenchyma of the lungs, wherein the therapeutic and pharmaceutical agents alleviate and prevent symptoms associated with asthma, atelectasis, bronchitis, COPD, emphysema, lung cancer, pneumonia and pulmonary edema.

Another object of the present invention is to provide improved methods and devices for the delivery of therapeutic and pharmaceutical agents to the small airways and parenchyma of the lungs, wherein the therapeutic and pharmaceutical agents alleviate and prevent symptoms associated with corticosteroid resistance (CR).

Another object of the present invention is to provide improved methods and devices for the delivery of therapeutic and pharmaceutical agents to the small airways and parenchyma of the lungs, wherein the therapeutic and pharmaceutical agents improve the development and regrowth of lung tissue.

A further object of the present invention is to provide improved methods and devices for the delivery of therapeutic and pharmaceutical agents to the small airways and parenchyma of the lungs, wherein the therapeutic and pharmaceutical agents improve the development, and regrowth of alveoli, and subsequent maintenance of the regrown alveoli.

Yet another object of the present invention is to provide improved methods and devices for the delivery of therapeutic and pharmaceutical agents to the small airways and parenchyma of the lungs, wherein the devices may be configured to deliver more than one therapeutic or pharmaceutical agent.

A further object of the present invention is to provide improved methods and devices for the delivery of therapeutic and pharmaceutical agents to the small airways and parenchyma of the lungs, wherein the devices may be configured to deliver more than one therapeutic or pharmaceutical agent such as those comprising, but not limited to, CR reversal agents, corticosteroids, bronchodilators, vitamin D (and active metabolites, vitamin D receptor agonists/partial agonists and equivalents thereof), and vitamin A (and active metabolites, vitamin A receptor agonists/partial agonists and equivalents thereof).

Another object of the present invention is to provide improved methods and devices for the delivery of therapeutic and pharmaceutical agents, which are targeted to be delivered to the small airways and parenchyma of the lungs, wherein the therapeutic and pharmaceutical agents comprise corticosteroids, muscarinic antagonists, macrolides, non-steroidal anti-inflammatory drugs (NSAIDs), bronchodilators and CR reversal agents.

Another object of the present invention is to provide improved methods and devices for the delivery of therapeutic and pharmaceutical agents to the small airways and parenchyma of the lungs, wherein the CR reversal agents comprise antioxidants, iNOS inhibitors, Phosphoinositide-3-kinase-6 inhibitors, p38 MAP kinase inhibitors, JNK inhibitors, MIF inhibitors, p-glycoprotein inhibitors, macrolides, calcineurin inhibitors, and vitamin D, synthetic vitamin D, vitamin D analogs, calcitiol and equivalents thereof.

Another object of the present invention is to provide improved methods and devices for the delivery of therapeutic and pharmaceutical agents to the small airways and parenchyma of the lungs, wherein the agents for improving pulmonary tissue growth and development comprise vitamin A, All Trans Retinoic Acid (ATRA), retinoic acid receptor (RAR) agonists and RAR selective alveolar growth agents and equivalents thereof.

Another object of the present invention is to provide improved methods and devices for the delivery of therapeutic and pharmaceutical agents to the small airways and parenchyma of the lungs, wherein the therapeutic and pharmaceutical agents comprise CR reversal agents and corticosteroids.

Another object of the present invention is to provide improved methods and devices for the delivery of therapeutic and pharmaceutical agents to the small airways and parenchyma of the lungs, wherein the therapeutic and pharmaceutical agents comprise budesonide, fluticasone, beclomethasone, flunisolide, triamcinolone, mometasone, any derivative or pharmaceutically acceptable salt thereof, or any other corticosteroid suitable for inhalation such as prodrugs (i.e. ciclesonide) or “soft” steroids which offer milder immunosuppression and fewer steroid side effects (i.e. loteprednol, fluorometholone).

Another object of the present invention is to provide improved methods and devices for the delivery of therapeutic and pharmaceutical agents to the small airways and parenchyma of the lungs, wherein the therapeutic and pharmaceutical agents comprise a combination of therapeutic agents selected from the group consisting of bronchodilators, CR reversal agent, a corticosteroid and pulmonary tissue growth and development agents such as vitamin A. Another object of the present invention is to provide improved methods and devices for the delivery of therapeutic and pharmaceutical agents to the small airways and parenchyma of the lungs, wherein the therapeutic and pharmaceutical agents comprise calcitriol, fluticasone and a bronchodilator.

A further object of the present invention is to provide improved methods and devices for the delivery of therapeutic and pharmaceutical agents to the small airways and parenchyma of the lungs, wherein the therapeutic and pharmaceutical agents alleviate and prevent symptoms associated with non-pulmonary diseases and malfunctions.

These and other objects, features and advantages of the present invention will become apparent after a review of the following detailed description of the disclosed embodiments and the appended claims.

### BRIEF DESCRIPTION OF THE DRAWINGS

FIG. 1 provides a schematic summarizing the experimental design of the effect of treatment of therapeutic compositions as described in Example 7 on smoke-exposed female mice.

### DETAILED DESCRIPTION OF PREFERRED EMBODIMENTS

The present invention may be understood more readily by reference to the following detailed description of the specific embodiments included herein. Reference is made to the accompanying drawings, which form a part hereof, and in which is shown, by way of illustration, various embodiments of the present disclosure. Although the present invention has been described with reference to specific details of certain embodiments thereof, it is not intended that such details should be regarded as limitations upon the scope of the invention. The entire text of the references mentioned herein are hereby incorporated in their entireties by reference including U.S. Provisional Patent Application Ser. No. 61/386,733 filed on Sep. 27, 2010, U.S. Provisional Patent Application Ser. No. 61/386,767 filed on Sep. 27, 2010, U.S. Provisional Patent Application Ser. No. 61/386,771 filed on Sep. 27, 2010, U.S. Provisional Patent Application Ser. No. 61/386,776 filed on Sep. 27, 2010. Also incorporated by reference are the following co-pending patent applications: U.S. patent application Ser. No. 12/785,082 filed on May 21, 2010, U.S. patent application Ser. No. 12/828,133 filed on Jun. 30, 2010 and U.S. patent application Ser. No. 12/985,158 filed on Jan. 5, 2011.

The methods and compositions of the present invention are particularly suited for the delivery of therapeutic and pharmaceutical agents to the lung. More specifically the methods and compositions of the present invention are particularly suited for the delivery of therapeutic and pharmaceutical agents to all the airway passages within the lung, including but not limited to, the bronchioles, the respiratory bronchioles, the alveolar ducts, the atria, the alveolar sacs, the alveoli (air sacs or clusters of cells). The present invention is further suited for the delivery of therapeutic and pharmaceutical agents to the circulatory system of the lung, including but not limited to, the pulmonary artery, pulmonary capillaries, pulmonary veins, bronchial arteries, and bronchial veins.

As discussed above, the unique features of the present invention enable the user of the inhaler to receive an effective dose of the desired pharmaceutical or therapeutic agent in an optimal manner. The inhalers used herein enable site-specific delivery of micronized dry powder or liquid medicaments in optimal fashion as a result of novel mechanical features that combine the dynamic properties of flow and inspiration, such that the user receives an appropriate therapeutic amount of the medicament.

The present invention satisfies the long felt need in the market for a device that has the capability to deliver medicaments in micronized form. The invention enables the delivery of medicaments having a particle size that is sufficiently small per mass median aerodynamic diameter (MMAD), and has the appropriate geometric standard deviation (GSD) to effectively target the airways of the lung, in particular the small airways of the lung. In addition, the internal mechanical features as described above enable the use of the device even by flow-restricted subjects for whom minimal flow rates are often problematic. The combination of features with the ergonomic design of the inhaler result in an easy to use device which is necessary for subjects having limited or restricted physical abilities (such as the elderly, very young, or infirm).

In one embodiment of the present invention the methods taught herein are directed to the treatment of pulmonary disease. The incidence of pulmonary diseases such as asthma, atelectasis, bronchitis, COPD, emphysema, lung cancer, pneumonia and pulmonary edema is steadily increasing and there exists a need for improved methods for delivering therapeutic agents to subjects suffering from such disease so that treatment and recovery is facilitated.

A complication of pulmonary diseases, especially COPD, is a condition often referred to as corticoidsteroid resistance (CR) wherein patients become poorly responsive to the anti-inflammatory actions of corticosteroids and consequently minimal clinical benefit is derived from such drugs. In some cases, it is thought that CR manifests when the administered corticosteroid agent does not reach the target areas of the lungs. Though not wishing to be bound by the following theory, additional contributing factors of CR are thought to include the impairment of histone deacetylase 2 (HDAC2) and/or the lack of IL-10 secretion from regulatory T-cells. In normal subjects, HDAC2 is involved in the switching off of inflammatory gene transcription. As a result of impairment, most likely resulting from cigarette smoking and oxidative stress, the function of HDAC2 is significantly reduced, gene transcription regulation is diminished, and ultimately synthesis of inflammatory proteins may proceed unchecked. IL-10 is known to play an important role in the downregulation of Th1 inflammatory cytokines and the promotion of regulatory T cells which help to control the inflammatory response. Certain studies have demonstrated that increasing IL-10 levels to normal levels, alleviates some of the problems associated with CR resistance.

There exists a crucial need in the art for an efficient method of delivering therapeutic agents to patients suffering from COPD, especially those who also have complications resulting from CR. Although practitioners skilled in the art recognize the possible causes of CR (i.e. inability to deliver therapeutic agents in deep lung tissue, impairment of HDAC function, disruption of IL-10 production), until the development of the present invention, no effective method or device had been created to meet the needs of such patients. The novel methods and devices of the present invention enable a patient for the first time to receive, via inhalation, an agent for CR reversal concurrently (or sequentially) with a corticosteroid. Although it has been established in the literature that certain agents possess the ability to improve the efficacy of corticosteroids in CR patients, an effective method to deliver and achieve beneficial levels in humans without inducing untoward side effects has not been devised until now.

The methods and devices of the present invention overcome problems associated with prior art methods such as those that result in undesirable side effects including diminished drug responsiveness due to non-targeted methods drug administration. In contrast, the novel features of the present invention enable controlled site delivery, namely the deposition of CR reversal agents in proximity to, or at the location of, corticosteroid deposition. Furthermore, the present invention addresses the complications that may arise from delivering more than one therapeutic agent wherein each agent displays dissimilar aerosol characteristics and deposition patterns. The invention satisfies the need for delivery of corticosteroids and agents for reversal of CR to the lungs where heightened local concentrations are obtained, systemic levels are minimized, and synergistic immunomodulating aspects of the two moieties are realized. In summary, the present invention provides novel methods and devices for pulmonary delivery of corticosteroids with agents for reversal of CR to a mammalian host, particularly a human patient, whereby a more significant and/or prolonged immunomodulatory response greater than that achieved by the corticosteroid alone is achieved.

It has been discovered that pulmonary co-administration of a corticosteroid with an agent for reversal of CR allows for lower dosage levels than would be necessary to achieve a similar pulmonary therapeutic response by other methods of delivery (i.e. oral delivery, intravenous delivery). This allows for reduction of systemic side effects of either or both agents. Using the inhalers of the present invention, co-administration allows direct targeting of the agent for reversal of CR to the site of action, since aerosol deposition of both agents occurs at the same region of the lung and throughout the lung compartments. Precise targeting of CR reversal agents allows for high local concentrations in the region of corticosteroid deposition, creating a microenvironment where corticosteroid activity is increased. Co-administration as described above offers a more patient compliant alternative to multiple-dosage medicaments and also provides greater therapeutic efficacy by supplying therapeutic levels of drug at the same tissue targets (particularly important for CR reversal). The unique features of this invention resulting in the direct administration of CR reversal agents to the lungs, enhance overall therapeutic effectiveness. For example, targeted drug delivery according to the methods herein result in advantages including, but not limited to, prolonged release resulting from slow dissolution, preferential lung tissue residence resulting from lipophilic interactions/cellular retention mechanisms, enhancement of pulmonary bioavailability resulting from avoidance of intestinal and hepatic metabolism, and enhancement of pulmonary bioavailability resulting from avoidance of poor absorption through the gastrointestinal wall.

In certain embodiments, the CR reversal agent comprises vitamin D, vitamin D analogs, synthetic vitamin D, vitamin D receptor agonists and antagonists, calcitriol, calcitol and equivalents thereof. Also included are CR reversal agents known to those skilled in the art, including, but not limited to, antioxidants, iNOS inhibitors, Phosphoinositide-3-kinase-δ inhibitors, theophylline, p38 MAP kinase inhibitors, JNK inhibitors, MIF inhibitors, p-glycoprotein inhibitors, macrolides, and calcineurin inhibitors.

The term “vitamin D” is intended to encompass not only vitamin D2 and vitamin D3, but any salt, metabolite, or derivative of vitamin D having immunoregulatory activity like vitamin D, and which is non-toxic and pharmacologically acceptable, for example, calcitriol.

One embodiment of the present invention comprises the administration of dry powder calcitriol via inhalation. Dosing ranges for such therapeutic administration may range from 0.0025 μg to 10 μg, from 0.05 μg to 5 μg, or from 0.1 μg to 2.5 μg. In addition, the mass median particle size of the calcitriol dry powder may range from 0.1 μm to 10 μm, from 0.25 μm to 5 μm, or from 0.5 μm to 4 μm. As would be evident to one skilled the art, appropriate dosing levels are ultimately determined by the size, weight, and age of the patient, as well as severity of symptoms to be treated. Nevertheless, one unique aspect of the present invention comprises low effective dosaging ranges. The unique methodology of the present invention enables the patients with pulmonary problems to receive compositions comprising vitamin D, including calcitriol, in low but highly effective doses. High vitamin D dosing levels can cause toxicity, however the effective delivery of low dosages of enables the patient to receive the beneficial effects of the therapeutic composition without potential toxicity. Until now, delivery of vitamin D compositions via inhalation has been discussed, but not actually reduced to practice. The inventors of the present invention have overcome problems such as toxicity and inability to achieve an effective concentration at the site of action, by developing stable, consistent dry powder formulations and effectively delivering them to the target lung region even for patients with compromised pulmonary function. Accordingly, though prior art studies and discussions make reference to vitamin D inhalation, successful therapeutic intervention comprising vitamin D inhalation was not accomplished until the present inventors demonstrated the delivery of vitamin D dry powder compositions by coupling suitable formulations with delivery via inhalation.

Pulmonary delivery of vitamin D via inhalation as described herein, to a patient, particularly a human patient, provides heightened and less variable pulmonary or systemic concentrations compared to those that could be achieved by other methods of administration. In addition, the direct administration of vitamin D to the lungs as described herein include but are not limited to prolonged release resulting from dose reduction, slow dissolution, preferential lung tissue residence resulting from lipophilic interactions, preferential lung tissue residence resulting from large molecular size, enhancement of bioavailability (as compared to oral administration) resulting from avoidance of absorption variability in the gut and reduction of intestinal and hepatic metabolism.

As referenced earlier, therapeutic effects of calcitriol have been documented in scientific studies for both pulmonary disease and for cancer. However, in such studies, calcitriol is utilized in very high doses in order for a positive effect to be attained. High dosing of calcitriol poses significant problems associated with toxicity due to hypercalcemia. Nevertheless calcitriol has the potential to function as an important and effective anti-inflammatory pharmaceutical, especially in the area of pulmonary disease such as COPD where there is no currently available effective anti-inflammatory therapeutic.

The present invention overcomes prior art problems by providing novel methods and compositions of calcitriol that are suitable for achieving therapeutic concentrations in the lung following low dose delivery via inhalation as opposed to oral intake which requires very extremely high doses to achieve the same lung concentrations and therefore risk significant toxicity. The methods and compositions of the present invention satisfy the long felt need in the art for a pulmonary disease therapeutic that not only results in the reduction inflammation and corticosteroid resistance, but also significantly minimizes toxicity.

In certain embodiments, dry powder calcitriol comprises a crystalline anhydrous form that is micronized to a particle size less than volume median particle size of approximately 2-8 microns and most preferably approximately 1-4 microns and is formulated with anhydrous lactose. In certain other dry powder embodiments, calcitriol may be prepared into a liquid calcitriol/lactose feedstock and processed using spray drying and/or ultrasonic evaporation processes to yield calcitriol-lactose fused crystals with a particle size less than volume median particle size of approximately 5 microns at a ratio of 1:10-1:1000, such fused crystals may be further formulated with anhydrous carrier lactose. In preferred embodiments, the formulations of calcitriol contain no triazoline adduct of pre-calcitriol and methylene calcitriol.

The dry powder calcitriol compositions as described above may be administered to patients via the use of an inhalation device. In one embodiment, such calcitriol compositions are administered using proprietary technology developed by MicroDose Therapeutx, Inc. (Monmouth Junction, N.J.). The compositions are packaged for unit dose delivery of 0.25-10.0 micrograms, 0.5-5.0 micrograms or 0.1-2.5 micrograms (or varying ranges thereof) in a dry powder inhaler (DPI) available from MicroDose Therapeutx, Inc. The combination of the unique formulation, particle size and delivery methodology results in effective therapeutic consequence: a reduction in corticosteroid resistance, and improvement in steroid therapy.

Delivery of the dry powder calcitriol compositions described herein via the inhalers developed by MicroDose Therapeutx, Inc. (as described in U.S. patent application Ser. Nos. 12/785,082, 12/828,133 and 12/985,158) accomplishes successful administration of appropriate doses to desired sites within the lung and pulmonary tissue. More specificially, calcitriol compositions may be delivered to small airways and parenchyma of the lungs for optimal results, namely reduction in corticosteroid resistance. Patients having compromised lung function benefit from the methods described herein as administration of therapeutic compositions are accomplished at a low flow rate. Patients having a breathing flow rate of even a minimal 10 L/min may utilize the inhalers described herein and dosing may be accomplished via tidal breathing irrespective of any specifically required breathing pattern. Moreover, the inhaler is designed to deliver drug in a single breathing maneuver at flow rates up to 30 L/min or over a series of tidal inhalations at peak flow rates less than 25 L/min.

In certain other embodiments, the administration of calcitriol may be optionally coupled with a pulmonary tissue growth or repair agent to take advantage of the anti-inflammatory action of calcitriol in offsetting selective pro-inflammatory action of the pulmonary tissue growth or repair agent.

In some embodiments, the administration of the calcitriol compositions as described above may be preceded by the administration of bronchodilator. In certain embodiments therapeutic intervention may involve the preliminary administration of a bronchodilator, followed by the administration of calcitriol optionally combined with a steroid such as fluticasone.

In some embodiments, the therapeutic regimen recommends implementation of the methods described herein at specific times of the day in order to optimize effectiveness based on natural biological variation in calcitriol metabolism. For example, since calcitriol exhibits diurnal variation with the low at around 0400 hr and a peak at 1600 hr followed by a decline in the evening, in a preferred embodiment, calcitriol dosing is recommended at night (preferably between 1800 hr and 2000 hr) to maximize local supplementation of calcitriol.

As mentioned above, in addition to corticosteroid resistance, another obstacle in pulmonary disease involves the destruction of pulmonary tissue. More specifically, the destruction of alveoli in COPD patients typically results in significant airspace enlargement with reduction of alveolar capillary exchange area. The alveoli become weakened and ruptured air sacs are unable to efficiently move oxygen from the air to the blood. Previous studies have demonstrated beneficial effects of agents that interact with the Retinoic Acid Receptor (RAR) on alveoli growth and regeneration, however, until the disclosure of the present invention herein, no effective therapeutic or administrative methods for inhalation of vitamin A (or related compounds thereof) were available. In contrast to currently available therapeutic methods, the methods herein involve administration of vitamin A via inhalation for controlled site delivery. As a result, vitamin A therapeutic compositions are delivered in close proximity to damaged alveoli for direct effect. More specifically, the delivery methods of the present invention achieve optimal delivery of vitamin A compositions at low doses thereby reducing unnecessary side effects such as skin reactions (for instance, mucucutaneous eruptions), and headache. The unique aspects of vitamin A composition delivery as claimed herein, comprise stable formulations and delivery systems optimized to administer less than 500 μg of active vitamin A compositions to patients with compromised lung function; such delivery systems coincide with tidal breathing and unlike currently available commercial devices, do not require coordination with a predetermined breathing patterns by a patient.

One embodiment of the present invention comprises the administration of dry powder vitamin A compositions via inhalation. Dosing ranges for such therapeutic administration may range from 0.05 μg to 10 μg, from 0.1 μg to 5 μg, or from 1 μg to 4 μg. In addition, the mass median particle size of the vitamin A dry powder may range from 0.1 μm to 10 μm, from 0.25 μm to 5 μm, or from 0.5 μm to 4 μm. As would be evident to one skilled the art, appropriate dosing levels are ultimately determined by the size, weight, and age of the patient, as well as severity of symptoms to be treated. Nevertheless, one unique aspect of the present invention comprises low effective dosing ranges. The unique methodology of the present invention enables the patients with pulmonary problems to receive compositions comprising vitamin A, including ATRA, in low but highly effective doses.

The vitamin A compositions of the present invention include ‘alveolar growth agents’ that promote the generation of new alveoli and are selected from agents that interact with the Retinoic Acid Receptor (RAR). Also included are ‘alveolar maintenance’ agents used in combination to maintain newly generated alveoli from being attacked by the progressive nature of COPD and to minimize unexpected deleterious effects of the aforementioned RAR therapy.

Various alveolar growth agents have been considered in clinical studies, however all such studies have been limited to methods of administration that do not include inhalation. These agents include but are not limited to ATRA, ATRA derivatives, RAR agonists, 13-cis Retinoic acid and RAR selective agonists i.e. palovarotene. In contrast, the methods of the present invention comprise compositions for inhalation with the goal of maximizing drug concentrations in the target (lung) and minimizing systemic exposure to the rest of the body.

The present invention further comprises alveolar maintenance agents including but not limited to: macrolides (Cyclosporine, Tacrolimus, Sirolimus, Clarithromycin, erythromycin, telithromycin, azithromycin), immunosuppressants (Mycophenolate sodium), anti-malarials (Hydroxychloroquine, mefloquine), NSAIDS (fenspiride), anti-oxidants (quercetin, curcumin compounds) and other vitamins/vitamin derivatives (vitamin D, C, E). The novel methods and compositions of the present invention comprise vitamin A formulations for inhalation which serve to minimize systemic exposure, provide effective amounts of both agents to the target organ (the lung) and avoid the complex systemic metabolism and bioavailability issues of ATRA and RAR agents.

The novel methods and compositions of the present invention overcome current problems in the prior art by achieving the effective delivery of therapeutic compositions via inhalation for alleviating and reducing symptoms associated with pulmonary disease. The compositions of the present invention comprise agents for reversing corticosteroid resistance such as vitamin D, calcitriol and equivalents thereof. In addition, the compositions of the present invention comprise alveolar growth and maintenance agents such as ATRA and erythromycin. Furthermore, the present invention may comprise a combination of therapeutics: certain embodiments may comprise agents for reversing corticosteroid resistance as well as agents for alveolar regrowth. Certain other embodiments may further comprise an alveolar maintenance agent. Additional embodiments may optionally comprise bronchodilating substances.

Certain preferred embodiments of the present invention comprise methods for the treatment of pulmonary disease comprising the administration of compositions comprising vitamin D and vitamin A via inhalation. More specifically, certain preferred embodiments comprise methods for the treatment of pulmonary disease, such as COPD, comprising the administration of compositions comprising calcitriol and ATRA via inhalation. Such embodiments overcome prior art problems associated with toxicity and achieve optimal therapeutic effect as a result of controlled site delivery.

In addition, certain preferred embodiments comprise methods of delivering calcitriol and ATRA in ratios from 1:50 to 1:500000 and more preferably from 1:500 to 1:50000. Also, plasma levels of calcitriol do not exceed 30 μg/mL above baseline levels in serum 4 hours following administration.

### Inhalers

Prior art inhalers are unable to deliver sufficiently micronized medicaments and as such, therapeutic intervention using such inhalers is not efficient or completely effective. In contrast, the inhalers of the present invention have the unique ability to deliver micronized medicaments to the lung airways, and more particularly to the small lung airways such that uptake of the medicament is accelerated and optimized. The specific embodiments and details of inhalers contemplated for use herein are described in detail in U.S. patent application Ser. No. 12/785,082 (Unites States Published Application No. 20100294287) filed on May 21, 2010, U.S. patent application Ser. No. 12/828,133 (Unites States Published Application No. 20110000481) filed on Jun. 1, 2010 and U.S. patent application Ser. No. 12/985,158 (Unites States Published Application No. 20110162642) filed on Jan. 5, 2011 and incorporated herein in its entirety.

In some embodiments, the methods of the present invention comprise devices wherein the improvements pertain to the internal dosing mechanics of the devices, the administration of individual doses, and also to the general delivery of the medicament. For example, one improvement pertains to the embodiment of an inhaler having a vibration element for aerosolizing medicament contained in a blister pack, wherein the inhaler is adapted to hold a plurality of individual blister packs which can be individually accessed and moved into an operative or dispensing position between the vibration element and a piercing element. The advantages of this construction include: simpler, more compact assembly for an inhaler containing a plurality of blister packs; and the ability to isolate and shield individual blister packs from the piercing element prior to use.

An additional improvement pertains to an inhaler comprising a compact size pharmaceutical delivery package including a unique dose drum formed into a cylinder and containing a plurality of dose compartments for containing individual doses. This improvement results in better therapeutic compliance by ensuring that the appropriate dose is delivered to a patient.

Another improvement involves the use of a specialized nebulizer that is particularly useful for pediatric patients and other patients with compromised physical abilities. The nebulizer contemplated herein utilizes a powder plume, that enables the delivery of aerosolized dry powders in much higher dose concentrations than are possible with liquid carried drugs. In addition, the generation of powder plume is independent of inhalation rate and inhalation timing and the use of the nebulizer results in reproducible and recordable pulmonary doses from pre-measured blister packs.

In accordance with the specific features described above, the inhaler of the present invention results in improved delivery of therapeutic or pharmaceutical agents by active device aerosol generation. The mechanism of delivery further utilizes pulmonary fluid as a delivery medium in order to deliver “through” airflow limited airways and delivery is accomplished while maintaining positive pressure within the lung. Such features overcome limitations that may have resulted because of airflow limitation caused by disease progression. Accordingly, efficient and effective drug delivery is accomplished regardless of narrowed, collapsed or otherwise compromised airway passages. For subjects such as those suffering from COPD with reduced inspiratory capacity and compromised lung function, therapeutic intervention using the presently described inhalers results in expedited relief and reduction of symptoms.

An additional advantage of the present invention the ability to deliver more than one therapeutic agent via inhalation without complications arising from disparate aerosolization profiles. The present inhalers overcome problems that result from dissimilar aerosol characteristics and deposition patterns. Accordingly, the present invention enables the delivery of more than one therapeutic agent, i.e. CR reversal agent, corticosteroid, pulmonary/alveolar growth agent, bronchodilator. In one embodiment of the present invention the option of administering a bronchodilating substance prior to the delivery of the therapeutic agent intended for deep lung delivery is provided. The bronchodilating substance may be delivered via the same inhaler device thereby increasing the subject's convenience, and ultimately improving therapeutic compliance. Also in accordance with the features described above, the methods and device of the present invention are particularly desirable because a concentrated plume of drug is delivered within the small volume of inhaled air at the onset of inspiration.

### Terms and Definitions

The terms “fine drug particles,” and “aerodynamic particle size” as used herein, mean particles having a size sufficiently small so as to be delivered to the airways of the lungs, and especially to the small airways. For optimal delivery to the lungs, the dry powder form of the therapeutic agents described herein preferably should be micronized, spray dried, or engineered to a maximum aerodynamic particle size in the range of 0.1 μm to 10 μm, from 0.25 μm to 5 μm, or from 0.5 μm to 4 μm.

As used herein, the term “agent for reversal of CR” is intended to encompass any agent that when administered at an effective level will increase the anti-inflammatory response induced by a corticosteroid. This term applies not only agents for reversal of CR, but any salt or derivative of said agent having activity to reverse CR, and which is non-toxic and pharmacologically acceptable.

As used herein, CR reversal agents, include but are not limited to, vitamin D, vitamin D analogs, synthetic vitamin D, vitamin D receptor agonists and antagonists, calcitol and equivalents thereof. Also included are CR reversal agents known to those skilled in the art. Including, but not limited to, antioxidants, iNOS inhibitors, Phosphoinositide-3-kinase-δ inhibitors, theophylline, p38 MAP kinase inhibitors, JNK inhibitors, MIF inhibitors, p-glycoprotein inhibitors, macrolides, and calcineurin inhibitors.

As used herein, the term “vitamin D” is intended to encompass vitamin D, vitamin D2, vitamin D3, vitamin D analogs, synthetic vitamin D, vitamin D receptor agonists and antagonists, calcitriol, calcitol and equivalents thereof.

As used herein, the term “vitamin A” is intended to encompass those agents that interact with Retinoic Acid Receptor (RAR) including but not limited ATRA, ATRA derivatives, RAR agonists, 13-cis Retinoic acid and RAR selective agonists for example, palovarotene.

As used herein, the term “alveolar growth agent” is intended to encompass any agent that promotes the growth of new alveoli via the retinoic acid receptor, and includes ATRA or RAR selective agent therapy.

As used herein, the term “alveolar maintenance agent” is intended to encompass any agent that when administered at an effective level will increase the anti-inflammatory response induced by COPD, COPDe and Emphysema and any undesirable effects of ATRA or RAR selective agent therapy. This term applies not only agents for alveolar maintenance, but any salt, hydrate, prodrug or derivative of said agent having similar activity, and which is non-toxic and pharmacologically acceptable.

As used herein, bronchodilating substances include, but are not limited to, beta2-agonists (short and long acting, LABA), long acting muscarinic antagonists (LAMA), anticholinergics (short acting), and theophylline (long acting). “Co-administered,” as used herein, means to deliver more than one pharmaceutical or therapeutic agent, for example, both corticosteroid and agent for reversal of CR as an aerosol within the same breath via the pulmonary route.

“An effective amount,” as used herein, is an amount of the pharmaceutical composition that is effective for achieving a desired therapeutic effect, including but not limited to bronchodilation, CR reversal, anti-inflammation, alveolar regrowth. For example, an effective amount of an agent for reversal of CR may comprise the specified amount of calcitriol, within a defined aerodynamic particle size range suitable for absorption in the lungs, that is able to reduce or eliminate the resistance to corticosteroids.

As used herein, “pharmaceutical” and “therapeutic” agents include but are not limited to any and all medicaments and pharmaceutical agents and formulations that may be administered for the treatment of pulmonary disease, including agents for preventing disease and including agents for maintaining improvement of disease condition. As used herein, such therapeutic and pharmaceutical agents include, but are not limited to, corticosteroids, muscarinic antagonists, macrolides, and non-steroidal anti-inflammatory drugs (NSAIDs), antioxidants, iNOS inhibitors, phosphoinositide-3-kinase-δ inhibitors, p38 MAP kinase inhibitors, JNK inhibitors, MIF inhibitors, p-glycoprotein inhibitors, macrolides, calcineurin inhibitors, and vitamin D, synthetic vitamin D, vitamin D analogs, calcitiol, vitamin A, All Trans Retinoic Acid (ATRA), retinoic acid receptor (RAR) agonists, RAR selective alveolar growth agents, budesonide, fluticasone, beclomethasone, flunisolide, triamcinolone, mometasone, ciclesonide, loteprednol, fluorometholone as well as any derivative, equivalent or pharmaceutically acceptable salt thereof.

A “pharmaceutical” or “therapeutic” composition as used herein, means a medicament for use in treating a patient, for example, an agent for reversal of CR in a dry powder form of a defined aerodynamic particle size prepared in a manner that is suitable for pulmonary administration to a patient. A pharmaceutical composition according to the invention may optionally, include a non-toxic pharmaceutically acceptable carrier. In certain embodiments “pharmaceutical” or “therapeutic” composition may comprise a singular entity (i.e. calcitriol alone), or a combination of compositions selected from the group consisting of CR reversal agents, anti-inflammatory agents, bronchodilators, alveolar growth agents, and others.

Other agents that may be delivered via the methods and inhaler described herein include, but are not limited to chemotherapeutics, angiogenesis inhibitors, kinase inhibitors, histone deacetylase inhibitors as well as other modifiers of epigenetic phenomena and proteosome inhibitors. Representative agents that may be used in the instant invention include, but are not limited to, the following; Aldeskeukin, Alemtuzumab, alitretinoin, allopurinol, altretamine, amifostine, anastrozole, arsenic trioxide, asparaginase, BCG Live, bexarotene capsules, bexarotene gel, bleomycin, busulfan intravenous, busulfan oral, calusterone, capecitabine, carboplatin, carmustine, carmustine with Polifeprosan 20 implant, celecoxib, chlorambucil, cisplatin, cladribine, cyclophosphamide, cytarabine, cytarabine liposomal, dacarbazine, dactinomycin actinomycin D, Darbepoetin alfa, daunorubicin liposomal, daunorubicin, daunomycin, Denileukin difitox, dexrazoxane, docetaxel, doxorubicin, doxorubicin liposomal, Dromostanolone propionate, Elliot's B Solution® (Orphan Medical Inc. Minnetonka, Minn.), epirubicin, Epoetin alfa, estramustine, etoposide phosphate, etoposide VP-16, exemestane, Filgrastim, floxuridine, fludarabine, fluorouracil, fulvestrant, gemcitabine, bemtuzumab ozogamicin, goserelin acetate, hydroxyurea, Ibritumomab Tiuxetan, idarubicin, ifosfamide, imatinib mesylate, Interferon alfa-2a, Interferon alfa-2b, irinotecan, letrozole, leucovorin, levamisole, lomustine CCNU, meclorethamine (nitrogen mustard), megestrol acetate, melphalan (L-PAM), mercaptopurine (6-MP), mesna, methotrexate, methoxsalen, mitomycin C, mitotane, mitoxantrone, MKC-1 nadrolone phenpropionate, Nofetumomab, Oprelvekin, oxaliplatin, paclitaxel, pamidronate, pegademase, Pegaspargase, Pegfilgrastim, pnetostatin, pipobroman, plicamycin (mithramycin), porfimer sodium, quinacrine Rasburicase, Rituximab, Sargramotim, streptozocin, talc, tamoxifen, temozolomide, teniposide (VM-26), testolactone, thioguanine (6-TG), thiotepa, topotecan, toremifene, Tositumomab, Trastuzumab, tretinoin (ATRA), Uracil Mustard, valrubicin, vinblastine, vincristine, vinorelbine and zoledronate.

It should be emphasized that the above-described embodiments of the present device and process, particularly, and “preferred” embodiments, are merely possible examples of implementations and merely set forth for a clear understanding of the principles of the disclosure. All these and other such modifications and variations are intended to be included herein within the scope of this disclosure and protected by the following claims. Therefore the scope of the disclosure is not intended to be limited except as indicated in the appended claims.

The following specific examples will illustrate the invention as it applies to the methods of treatment using the inhaler. It will be appreciated that other examples, including minor variations in procedures will be apparent to those skilled in the art, and that the invention is not limited to these specific illustrated examples.

### Example 1

### Controlled Site Delivery of Corticosteroid and Corticosteroid Resistant Agents via Inhalation

Inhaled corticosteroids (ICS) mometasone furoate or fluticasone furoate are prepared with volume median particle size of less than 5 microns. Calcitriol (1, 25-Dihydroxycholecalciferol) is also prepared in crystalline form and subsequently micronized to a volume median particle size of less than 5 microns. The ICS's are incorporated at appropriately 30-50% of the commercial ICS dose when administered via a passive dry powder inhaler, due to the efficiency of the invention delivered by a dry powder inhaler (DPI) available from MicroDose Therapeutx, Inc. One preferred embodiment utilizes an ICS dosed once daily, i.e. mometasone furoate or fluticasone furoate, to coincide with a once daily dose of the vitamin D receptor agonist. This combination product is designed to reverse corticoidsteroid resistance (CR) by adding the protective anti-inflammatory effects of calcitriol with the local anti-inflamatory effects of these ICS's. The inhaler is operated at 15 L/min and for both medicaments, the aerosol performance with a fine particle fraction (% of particles exiting the inhaler that are less than 5.8 microns) is less than or equal to_{—}45% with at least 10% of particles in the less than 2.1 micron size range when tested with a next generation Impactor.

### Example 2

### Controlled Site Delivery of Corticosteroid and Corticosteroid Resistant Agents via Inhalation

The ICS of Example 1, in crystalline form, are micronized to a maximum particle size of about 5 microns. A dry powder unit dose containing clinically effective doses of either ICS is blended with 1000 micrograms lecithin and packaged for delivery in a dry powder inhaler (DPI) available from MicroDose Therapeutx, Inc. This combination is designed to spread into alveolar fluid and treat lung parenchyma through partially occluded small airways.

### Example 3

### Controlled Site Delivery of Corticosteroid and Corticosteroid Resistant Agents via Inhalation

The ICS formulation from Example 1 or 2 is combined with albuterol sulfate in crystalline form separately micronized to a maximum particle size of about 5 microns. Delivery from a multiple dose dry powder inhaler (DPI) available from MicroDose Therapeutx, Inc. (Monmouth, N.J.) leverages the short acting bronchodilation of albuterol to allow deeper penetration of the ICS into the lung parenchyma.

### Example 4

### Calcitriol Compositions for Inhalation

Calcitriol is a synthetic vitamin D analog and has been used as a pharmaceutical as well as a nutraceutical. It is the synthetic version of a vitamin D metabolite that naturally occurs in the body. Calcitriol in the crystalline anhydrous form is micronized to a particle size less than volume median particle size of 4 microns and is formulated with anhydrous lactose. The resulting formulation has a residual moisture of less than 1% and loss of drying of less than 1.5%. The powder is packaged for unit dose delivery of 0.5-2.5 micrograms in a dry powder inhaler (DPI) available from MicroDose Therapeutx, Inc. (Monmouth, N.J.). The formulation is contained within a blister packaged under inert gas blanket (e.g. Nitrogen) within an aluminum-polymer laminate heat sealed blister to protect the formulation from moisture, light and oxygen. The inhaler is operated at 15 L/min and yields an aerosol performance with a fine particle fraction (% of particles exiting the inhaler that are less than 5.8 microns) of at least 50% with at least 10% of particles in the less than 2.1 micron size range when tested with a next generation Impactor. The formulation of calcitriol contains no triazoline adduct of pre-calcitriol and methylene calcitriol.

Use of the aforementioned calcitriol composition and administration via the MicroDose Therapeutx, Inc. DPI results in optimal delivery of the composition to the affected areas of the lung and enables reduced corticosteroid resistance.

### Example 5

### Calcitriol-Lactose Compositions for Inhalation

Calcitriol is prepared into a liquid calcitriol/lactose feedstock and processed using spray drying and/or ultrasonic evaporation processes to yield calcitriol-lactose fused crystals with a particle size less than volume median particle size of 5 microns at a ratio of 1:10-1:1:1000. The aforementioned fused crystals can be further formulated with anhydrous carrier lactose. The resulting formulation has a residual moisture of less than 1% and loss of drying of less than 1.5%. The powder is packaged for unit dose delivery of 0.5-2.5 micrograms in a dry powder inhaler (DPI) available from MicroDose Therapeutx, Inc. (Monmouth, N.J.).

Use of the aforementioned calcitriol composition and administration via the MicroDose Therapeutx, Inc. DPI results in optimal delivery of the composition to the affected areas of the lung and enables reduced corticosteroid resistance.

### Example 6

### ATRA and Calcitriol Compositions for Inhalation

All Trans Retinoic Acid (ATRA) is prepared in crystalline form and subsequently micronized to a volume median particle size of less than 5 microns. Calcitriol (1, 25-Dihydroxycholecalciferol) is also prepared in crystalline form and subsequently micronized to a volume median particle size of less than 5 microns.

The powder is packaged for unit dose delivery of 10-1000 micrograms of ATRA and 0.5-2.5 micrograms of calcitriol, formulated in an inhalation-grade anhydrous lactose blend in a dry powder inhaler (DPI) available from MicroDose Therapeutx, Inc. This combination product is designed to maximize alveolar regrowth and maintenance potential by adding the protective anti-inflammatory effects of calcitriol with alveolar regrowth induction of ATRA. The inhaler is operated at 15 L/min and for both medicaments, the aerosol performance with a fine particle fraction (% of particles exiting the inhaler that are less than 5.8 microns) is less than or equal to 45% with at least 10% of particles in the less than 2.1 micron size range when tested with a next generation Impactor.

Administration of the combination ATRA and calcitriol formulation results in reduced corticosteroid resistance and improved alveolar growth and maintenance.

### Example 7

### Effect of Treatment with Test Articles A, B, C, D, E, F, G, H and I in Cigarette Smoke-Exposed Female C3H/HeN Mice (3 weeks of exposure)

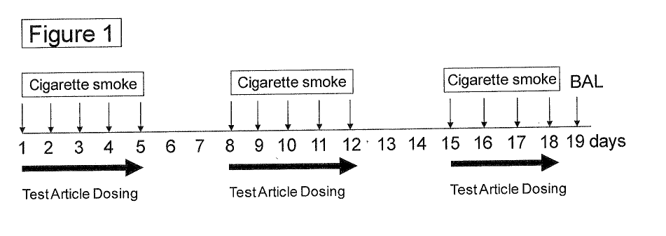

This study (see FIG. 1 and Table 1) will evaluate the efficacy of Test Articles A, B, C, D, E, F, G, H and I on inflammatory endpoints in female C3H/HeN mice (6-8 weeks of age on arrival and 8-10 weeks of age at start of exposure) exposed to filtered air (FA) or cigarette smoke (CS) for 6 hours per day, 5 days per week for 3 weeks (except for the third week where exposure will be for only 4 days). Mice will be exposed to FA sham (no vehicle), FA plus vehicle, CS plus vehicle, and CS plus intratracheal (IT) delivered Test Articles A, B, C, D, E, F, G, H and (doses to be determined). Dosing of Test Articles will begin the 1^{st} day of CS exposure (see FIG. 1) and will be administered q.d. (immediately before CS exposure) for days 1-5, 8-12, and 15-18. Some animals may be stagger-started as necessary to accommodate dosing, necropsy and sample processing. At the end of the study, mice will be euthanized and blood collected for blood gas analysis and plasma isolation. Bronchoalveolar lavage (BAL) will be performed on the lungs using three aliquots of PBS. BAL fluid will be analyzed at LRRI for total cell counts and differentials (macrophages, neutrophils, lymphocytes and eosinophils will be counted on cell differential slides). Lung lobes (lavaged) and cell-free BAL supernatant will be snap-frozen and stored at −80° C. Lung tissue (lavaged) will be analyzed at LRRI for IL-6, IL-10, IL1-α, IL1-β, eotaxin, RANTES, MCP-1, MIP-1α, TNF-α, KC, IL-β, GM-CSF, IP-10, and IFN-γ using Luminex. Lung tissue will also be analyzed for HDAC2. Plasma and cell-free BAL supernatant will be stored at −80° C. and sent to the sponsor.

TABLE 1
Treatment Groups for Cigarette Smoke-Induced Pulmonary Inflammation

| | | Whole-Body | | | | |
|---|---|---|---|---|---|---|
| | | smoke | | | | |
| Group No./ | Animal | exposure | | | Delivery | |
| Descriptor | # | (5 days/week) | Air | Treatment | Route/Frequency | Tissues Collected/Endpoints |
| 1. Sham | 8 | − | + | None (IT | IT | Blood collected for blood gas |
| | | | | bolus of air) | | analysis followed by processing to |
| | | | | | | plasma. Plasma sent to sponsor. |
| | | | | | | Whole lung - BAL (total cells and |
| | | | | | | differentials). Cell-free BAL |
| | | | | | | supernatant collected and sent to |
| | | | | | | sponsor. |
| | | | | | | Lung lobes - snap frozen |
| | | | | | | individually after lavage - |
| | | | | | | cytokines, chemokines and HDAC2. |
| 2. Vehicle | 8 | − | + | Vehicle | IT/q.d. | Blood collected for blood gas |
| | | | | | (5 days/week) | analysis followed by processing to |
| | | | | | | plasma. Plasma sent to sponsor. |
| | | | | | | Whole lung - BAL (total cells and |
| | | | | | | differentials). Cell-free BAL |
| | | | | | | supernatant collected and sent to |
| | | | | | | sponsor. |
| | | | | | | Lung lobes - snap frozen |
| | | | | | | individually after lavage - |
| | | | | | | cytokines, chemokines and HDAC2. |
| 3. Vehicle | 8 | + | − | Vehicle | IT/q.d. | Blood collected for blood gas |
| | | | | | (5 days/week) | analysis followed by processing to |
| | | | | | | plasma. Plasma sent to sponsor. |
| | | | | | | Whole lung - BAL (total cells and |
| | | | | | | differentials). Cell-free BAL |
| | | | | | | supernatant collected and sent to |
| | | | | | | sponsor. |
| | | | | | | Lung lobes - snap frozen |
| | | | | | | individually after lavage - |
| | | | | | | cytokines, chemokines and HDAC2. |
| 4. Test Article A | 8 | + | − | Calcitriol | IT/q.d. | Blood collected for blood gas |
| | | | | Low Dose | (5 days/week) | analysis followed by processing to |
| | | | | | | plasma. Plasma sent to sponsor. |
| | | | | | | Whole lung - BAL (total cells and |
| | | | | | | differentials). Cell-free BAL |
| | | | | | | supernatant collected and sent to |
| | | | | | | sponsor. |
| | | | | | | Lung lobes - snap frozen |
| | | | | | | individually after lavage - |
| | | | | | | cytokines, chemokines and HDAC2. |
| 5. Test Article B | 8 | + | − | ATRA | IT/q.d. | Blood collected for blood gas |
| | | | | | (5 days/week) | analysis followed by processing to |
| | | | | | | plasma. Plasma sent to sponsor. |
| | | | | | | Whole lung - BAL (total cells and |
| | | | | | | differentials). Cell-free BAL |
| | | | | | | supernatant collected and sent to |
| | | | | | | sponsor. |
| | | | | | | Lung lobes - snap frozen |
| | | | | | | individually after lavage - |
| | | | | | | cytokines, chemokines and HDAC2. |
| 6. Test Article C | 8 | + | − | Calictriol | IT/q.d. | Blood collected for blood gas |
| | | | | High Dose | (5 days/week) | analysis followed by processing to |
| | | | | | | plasma. Plasma sent to sponsor. |
| | | | | | | Whole lung - BAL (total cells and |
| | | | | | | differentials). Cell-free BAL |
| | | | | | | supernatant collected and sent to |
| | | | | | | sponsor. |
| | | | | | | Lung lobes - snap frozen |
| | | | | | | individually after lavage - |
| | | | | | | cytokines, chemokines and HDAC2. |
| 7. Test Article D | 8 | + | − | Dexamethasone | IT/q.d. | Blood collected for blood gas |
| | | | | | (5 days/week) | analysis followed by processing to |
| | | | | | | plasma. Plasma sent to sponsor. |
| | | | | | | Whole lung - BAL (total cells and |
| | | | | | | differentials). Cell-free BAL |
| | | | | | | supernatant collected and sent to |
| | | | | | | sponsor. |
| | | | | | | Lung lobes - snap frozen |
| | | | | | | individually after lavage - |
| | | | | | | cytokines, chemokines and HDAC2. |
| 8. Test Article E | 8 | + | − | Dexamathasone + | IT/q.d. | Blood collected for blood gas |
| | | | | Calcitriol | (5 days/week) | analysis followed by processing to |
| | | | | | | plasma. Plasma sent to sponsor. |
| | | | | | | Whole lung - BAL (total cells and |
| | | | | | | differentials). Cell-free BAL |
| | | | | | | supernatant collected and sent to |
| | | | | | | sponsor. |
| | | | | | | Lung lobes - snap frozen |
| | | | | | | individually after lavage - |
| | | | | | | cytokines, chemokines and HDAC2. |
| 9. Test Article F | 8 | + | − | Calcitriol + | IT/q.d. | Blood collected for blood gas |
| | | | | ATRA | (5 days/week) | analysis followed by processing to |
| | | | | | | plasma. Plasma sent to sponsor. |
| | | | | | | Whole lung - BAL (total cells and |
| | | | | | | differentials). Cell-free BAL |
| | | | | | | supernatant collected and sent to |
| | | | | | | sponsor. |
| | | | | | | Lung lobes - snap frozen |
| | | | | | | individually after lavage - |
| | | | | | | cytokines, chemokines and HDAC2. |

We claim:

1. A method for treating pulmonary disease comprising the use of an inhaler for administrating pharmaceutical agents.

2. The method of claim 1, wherein the pharmaceutical agent comprises bronchodilators, corticosteroids, corticosteroid reversal agent, and alveolar growth agents.

3. The method of claim 2, wherein the bronchodilators comprise long-acting beta₂-agonists or long acting muscarinic antagonists.

4. The method of claim 2, wherein the corticosteroids comprise budesonide, fluticasone, beclomethasone, flunisolide, triamcinolone, ciclesonide, loteprednol, fluorometholone, and derivatives or pharmaceutically acceptable salts thereof.

5. The method of claim 2, wherein the corticosteroid reversal agent comprises vitamin D, synthetic vitamin D, vitamin D analogs, vitamin D receptor agonists, vitamin D receptor partial agonists, calcitriol, calcitiol, antioxidants, iNOS inhibitors, Phosphoinositide-3-kinase-δ inhibitors, p38 MAP kinase inhibitors, JNK inhibitors, MIF inhibitors, p-glycoprotein inhibitors, macrolides, calcineurin inhibitors, and equivalents thereof.

6. The method of claim 2, wherein the alveolar growth agent comprises vitamin A, All Trans Retinoic Acid (ATRA), retinoic acid receptor (RAR) agonists and RAR selective alveolar growth agents, RAR selective agonists, palovarotene and equivalents thereof.

7. The method of claim 1, wherein the pulmonary disease comprises asthma, atelectasis, bronchitis, COPD, emphysema, lung cancer, pneumonia and pulmonary edema.

8. The method of claim 1, wherein the pulmonary disease comprises COPD and the pharmaceutical agents comprise a corticosteroid reversal agent and a corticosteroid.

9. The method of claim 8, wherein the corticosteroid reversal agent comprises calcitriol, and the corticosteroidand comprises fluticasone.

10. The method of claim 9, optionally comprising a bronchodilator or an alveolar growth agent.

11. A method for treating COPD comprising the use of an inhaler for administrating pharmaceutical agents.

12. The method of claim 11, wherein the pharmaceutical agents comprise bronchodilators, corticosteroids, corticosteroid reversal agent, and alveolar growth agents.

13. The method of claim 12, wherein the corticosteroid reversal agent comprises vitamin D, synthetic vitamin D, vitamin D analogs, vitamin D receptor agonists, vitamin D receptor partial agonists, calcitriol, calcitiol, antioxidants, iNOS inhibitors, Phosphoinositide-3-kinase-δ inhibitors, p38 MAP kinase inhibitors, JNK inhibitors, MIF inhibitors, p-glycoprotein inhibitors, macrolides, calcineurin inhibitors, and equivalents thereof.

14. The method of claim 13, wherein calcitriol comprises a crystalline anhydrous form.

15. The method of claim 13, wherein calcitriol comprises calcitriol-lactose fused crystals.

16. The method of claim 12, wherein alveolar growth agent comprises vitamin A, All Trans Retinoic Acid (ATRA), retinoic acid receptor (RAR) agonists and RAR selective alveolar growth agents, RAR selective agonists, palovarotene and equivalents thereof.

17. The method of claim 12, further comprising alveolar maintenance agents.

18. The method of claim 17, wheren the alveolar maintenance agents comprise macrolide, cyclosporine, tacrolimus, sirolimus, clarithromycin, erythromycin, telithromycin, azithromycin, immunosuppressants, mycophenolate sodium, anti-malarials, hydroxychloroquine, mefloquine, NSAIDs, fenspiride, anti-oxidants quercetin, curcumin compounds, vitamin D, vitamin C, and vitamin E.

19. The method of claim 11, wherein the pharmaceutical agents comprise calcitriol and ATRA.

20. The method of claim 19, further comprising alveolar maintenance agents.
